(19) United States
(12) Patent Application Publication
(10) Pub. No.: US 2026/0151562 A1
(43) Pub. Date: Jun. 4, 2026

(54) ANTI-ROTATION MECHANISM IN FLUID DELIVERY DEVICES

(71) Applicant: Embecta Corp., Parsippany, NJ (US)

(72) Inventor: Alessandro E. Pizzochero, Chelmsford, MA (US)

(73) Assignee: Embecta Corp., Parsippany, NJ (US)

(21) Appl. No.: 19/134,134

(22) PCT Filed: Nov. 30, 2023
(86) PCT No.: PCT/US2023/081938

§ 371 Date: May 29, 2025

Related U.S. Application Data
(60) Provisional application No. 63/385,499, filed on Nov. 30, 2022.

(51) Int. Cl. A61M 5/145; A61M 5/142

(52) U.S. Cl. CPC A61M 5/14566; A61M 5/14248; A61M 2005/14252

(57) ABSTRACT

Provided herein are fluid delivery systems including an inner screw having a pusher disposed at a distal end of the inner screw to engage and linearly translate a plunger within a reservoir to dispense a medication from the reservoir; an outer screw concentrically surrounding the inner screw, wherein an inner surface of the outer screw engages with an outer surface of the inner screw such that a rotational movement of the outer screw advances the inner screw from a nested configuration to an extended configuration; and a screw support configured to engage with the outer screw; and an anti-rotation sleeve (ARS) engaged with the screw support and the inner screw, such that, when the outer screw is rotated, the inner screw is advanced without rotating. The fluid delivery systems can be used in connection with fluid delivery devices.

### RELATED APPLICATIONS

This application claims the benefit of and priority to U.S. Provisional Ser. No. 63/385,499 , filed Nov. 30, 2022, which is incorporated herein by reference in its entirety.

### FIELD

This disclosure relates to anti-rotation mechanisms in fluidic pumps for delivery of therapeutic compounds.

### BACKGROUND

Fluidic pumps are ubiquitous in delivering insulin for diabetes management. Insulin pumps help users keep their blood glucose levels within target ranges based on individual need, and must provide continuous infusion at varying rates to mimic the behavior of the pancreas.

Typical pump designs for fluid delivery are challenged by the need achieve small size, low power consumption, accurate delivery, high reliability, and low manufacturing costs. In addition, drug delivery patch pump designs cannot impact drug quality. For example, the materials used for pump mechanism components that contact the delivered fluid cannot present biocompatibility problems. Screw-driven mechanisms have been used in pumps for dispensing medication, but still require improvement.

### SUMMARY

The above and other problems are overcome by illustrative embodiments.

In some aspects, the techniques described herein relate to a fluid delivery system, including: an inner screw having a pusher disposed at a distal end of the inner screw to engage and linearly translate a plunger within a reservoir to dispense a medication from the reservoir; an outer screw concentrically surrounding the inner screw, wherein an inner surface of the outer screw engages with an outer surface of the inner screw such that a rotational movement of the outer screw advances the inner screw from a nested configuration to an extended configuration; a screw support configured to engage with the outer screw; and an anti-rotation sleeve (ARS) engaged with the screw support and the inner screw, such that, when the outer screw is rotated, the inner screw is advanced without rotating.

In some aspects, the techniques described herein relate to a fluid delivery system, including: a reservoir including an outlet port at a distal end, and plunger movable along a longitudinal axis of the reservoir, the plunger configured to provide a seal with respect to inner walls of the reservoir; and a plunger driver, the plunger driver including: an inner screw being configured to advance the plunger along the longitudinal axis of the reservoir; an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir; a screw support configured to be inserted into the reservoir at a proximal end of the reservoir and configured to engage with the outer screw; and an anti-rotation sleeve (ARS) engaged with the screw support and the inner screw, such that, when the outer screw is rotated, the inner screw is advanced without rotating.

In some aspects, the techniques described herein relate to a fluid delivery device, the fluid delivery device including: a housing; a reservoir disposed within the housing, the reservoir including an outlet port at a distal end, and plunger movable along a longitudinal axis of the reservoir, the plunger configured to provide a seal with respect to inner walls of the reservoir; a plunger driver, the plunger driver including: an inner screw being configured to advance the plunger along the longitudinal axis of the reservoir; an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir; a screw support configured to be inserted into the reservoir at a proximal end of the reservoir and configured to engage with the outer screw; and an anti-rotation sleeve (ARS) engaged with the screw support and the inner screw, such that, when the outer screw is rotated, the inner screw is advanced without rotating; a drive nut concentrically surrounding and engaging the outer screw, wherein the drive nut is configured to be driven by an actuator to rotate the outer screw; and a fluid path including a proximal end in fluid communication with the outlet port of the reservoir and a distal end configured to be inserted into a patient.

In some aspects, the fluid delivery device is a patch pump or a medication pen.

### BRIEF DESCRIPTION OF THE DRAWINGS

The features of the disclosure are set forth with particularity in the appended claims. The patent or application file contains at least one drawing executed in color. Copies of this patent or patent application publication with color drawing(s) will be provided by the Office upon request and payment of the necessary fee. A better understanding of the features of the present disclosure will be obtained by reference to the following detailed description that sets forth illustrative embodiments, in which the principles of the disclosure are utilized, and the accompanying drawings of which:

FIG. 1 is an embodiment of a perspective view of a fluid delivery assembly;

FIG. 2 illustrates an exploded view of an embodiment of a plunger driver;

FIGS. 3A-3B illustrate assembled and cross-sectional views, respectively, of the plunger driver of in FIG. 2;

FIGS. 4A-4B illustrate perspective views of the plunger driver of in FIG. 2 in a partially extended state and a fully extended state, respectively;

FIG. 5 illustrates a side view of the plunger driver of in FIG. 2 in a fully extended state;

FIG. 6 is an exploded view of an exemplary delivery device according to some embodiments; and

FIGS. 7A-7B are perspective view and a top perspective view with the cover removed, respectively, of a wearable fluid delivery device constructed in accordance with an example embodiment.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides an anti-rotation system for enabling a telescoping screw mechanism to work with a round cross-section reservoir in fluid delivery devices. Telescoping screw mechanisms are used for advancement of a plunger into a fluid reservoir for controlled dispensing of fluids. However, they require anti-rotation features to enable movement between a nested configuration and an extended configuration, otherwise the plunger is prevented from advancing through the reservoir. The driver system described herein overcomes this challenge and provides anti-rotation capability to the telescoping screw mechanism.

Illustrative embodiments in this disclosure relate generally to pump mechanisms or fluid delivery assemblies for use in fluid delivery devices such as wearable medication infusion patches. Illustrative embodiments relate generally to nesting telescopic screws with anti-rotation sleeves for controllably extending or retracting a plunger driver in a syringe barrel-type reservoir that do not affect reservoir volume to ensure biocompatibility, that are fully retractable outside reservoir, and engage with the plunger for anti-rotation control. Telescoping screw-driven mechanisms have been designed in pumps for dispensing medication, but rely on restraining rotation in one of the screws in order to generate extension, otherwise the system would simply spin without advancing. This is required as the opposite end of the telescoping screw is fixed with the body of the device and provides a reference for the rotation and advance of one of the screws, In a simple embodiment, a non-circular shape of the reservoir can be used to create anti-rotation and thus to enable extension of a plunger from a nested position. This disclosure relates to anti-rotation mechanisms for circular or round shaped reservoirs.

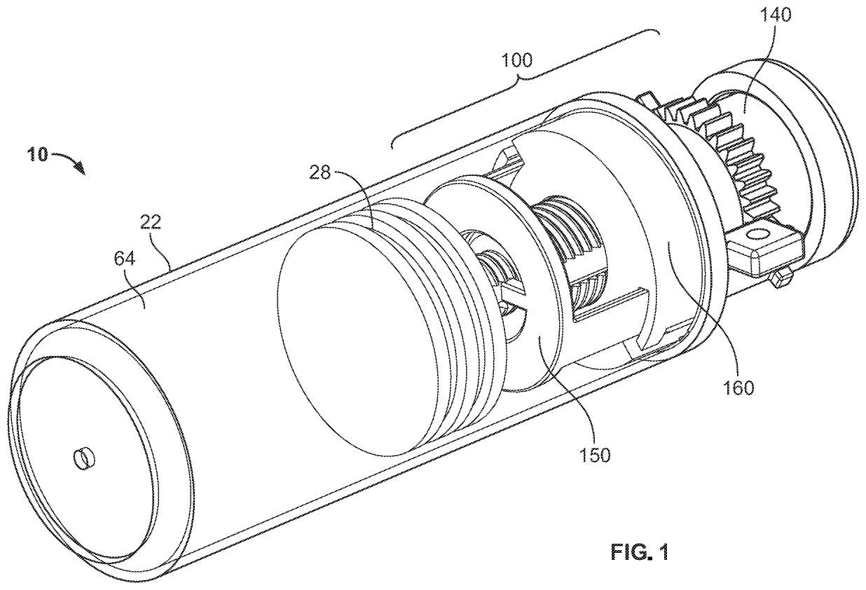

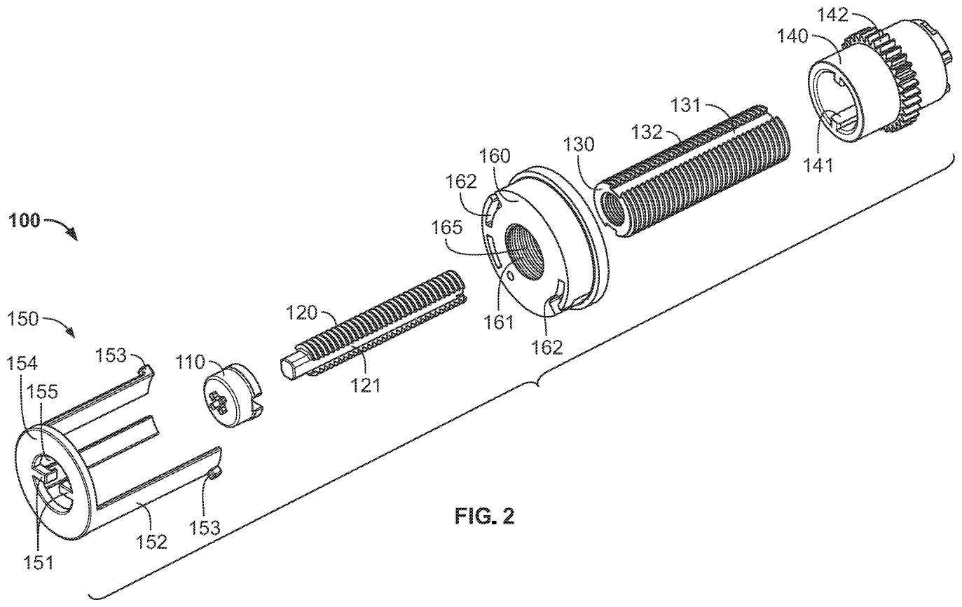

FIG. 1 is a perspective view of the fluid delivery assembly 10 to be used in a fluid delivery device as will be described below. The fluid delivery assembly 10 includes a plunger driver 100 configured to move a plunger 28 within a reservoir 22 to dispense a fluid to a user, such as, for example, a patient, a healthcare professional, or some other operator. In some embodiments, the fluid comprises a liquid medicament, such as, for example, insulin. In some embodiments, the plunger driver 100 includes a plurality of telescopic nested screws and an anti-rotation mechanism, to be described in more detail below with respect to FIGS. 2-5, and a drive nut 140. The drive nut 140 may be rotated via a gear train connected to a motor. The plunger driver 100 may be provided at a proximal end of the reservoir 22. In some embodiments, the reservoir 22 is circular or rounded, and the components of the fluid delivery are shaped and sized to fit in a round or circular reservoir. In some embodiments, the reservoir 22 comprises an inlet port such that the reservoir can be filled with a fluid. The plunger 28 is movable along a longitudinal axis of the reservoir 22 as the plunger driver 100 is engaged with the drive nut 140. In some embodiments, the plunger 28 is configured to provide a seal with respect to inner walls of the reservoir 22 to prevent fluid provided in a fluid chamber portion 64 defined on a first side of the plunger 28.

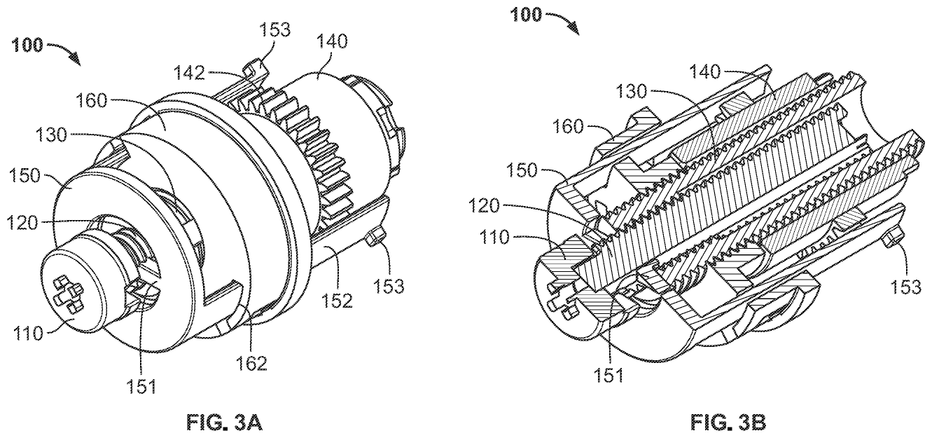

FIGS. 2-5 illustrate the plunger driver 100 according to some aspects of this disclosure. FIG. 2 illustrates an exploded view of the plunger driver 100. FIGS. 3A-3B illustrate assembled and cross-sectional views, respectively, of the plunger driver 100. In some embodiments, the plunger driver 100 comprises a pusher 110 mounted at a proximal end of an inner screw 120, wherein the pusher 110 engages the plunger 28. In some embodiments, the plunger driver 100 comprises an outer screw 130 concentrically surrounding the inner screw 120. In some embodiments, an inner surface of the outer screw 130 comprises a first mating surface, wherein the first mating surface may be threads. In some embodiments, an outer surface of the inner screw 120 comprises a second mating surface, wherein the second mating surface may be threads. In some embodiments, the first mating surface engages the second mating surface, such that at least one of the first mating surface and second mating surface rotate with respect to the other. In some embodiments, at least one of inner screw 120 and outer screw 130 rotates with respect to the other, wherein the second mating surface is configured to cooperate with the first mating surface to advance the inner screw 120 within the outer screw 130 when the outer screw 130 is rotated.

In some embodiments, the plunger driver 100 comprises the drive nut 140 concentrically surrounding and engaging the outer screw 130. In this manner, the plunger driver may be operated by applying torque on the drive nut. In some embodiments, drive nut 140 comprises a one or more inner protrusion 141. In some embodiments, outer screw 130 comprises one or more outer slot 131. In some embodiments, the one or more inner protrusion 141 engages the one or more outer slot 131 to achieve engagement between the drive nut 140 and outer screw 130. In some embodiments, the drive nut may be connected to a drive mechanism to operate the plunger driver 100. For example, the drive mechanism can be gears, ratchets, or other methods of inducing rotation from a motor. The drive nut 140 further comprises outer teeth 142. In some embodiments, the outer teeth 142 may be configured to engage an output gear of a motor gearbox. The outer teeth may be configured to directly engage the motor. In other words, this engagement may be achieved through direct engagement through a gear train. In some embodiments, the outer teeth are configured to indirectly engage the motor through at least one of a gear train and an outermost drive screw. In some embodiments, the engagement can be a coupling without a gear but with other torque coupling methods such as belts and pulleys. In some embodiments, the engagement can be axial coupling between a gear-less drive nut and input rotary energy. As an example, this engagement could be used in a medication pen and receive rotary motion from either the press of a button or other means to introduce motion into a pen (torsional springs, in-line motors with planetary gears, etc.).

In some embodiments, the plunger driver 100 comprises an anti-rotation sleeve (ARS) 150 engaged with a screw support 160 and the inner screw 120, such that, when the drive nut 140 is rotated, the pusher 110 moves the plunger 28 without rotating from a nested configuration that does not extend into the reservoir 22 to an extended configuration that extends from a proximal end of the reservoir 22 into the reservoir 22. The screw support 160 may further function as a cap for reservoir 22. In some embodiments, a face of the screw support 160 is circular to fit into a circular reservoir. In some embodiments, outer screw 130 comprises outer surface threading 132. In some embodiments, the screw support 160 comprises an opening 165 sized to receive the outer screw 130. In some embodiments, the opening 165 of the screw support 160 comprises an inner surface threading 161, wherein the outer surface threading 132 is configured to engage the inner surface threading 161. This engagement provides a fixed reference for rotation between the outer screw 130 and screw support 160 such that outer screw 130 may advance through the opening 165 of the screw support 160. In some embodiments, screw support 160 further comprises a vent hole that is sized to allow for faster air flow. Such faster air flow may be necessary during a higher rate of delivery when any other openings in support 160 are smaller, and/or when the screw support 160 is sealed.

In reference to FIG. 2, in some embodiments, the ARS 150 comprises a base 154 with an opening 155 sized to receive the inner screw 120. In some embodiments, the base 154 of the ARS 150 is circular in shape to fit into a circular reservoir. In some embodiments, the ARS 150 comprises a one or more protrusion 151 extending inwardly into the opening 155. In some embodiments, the inner screw 120 comprises a one or more slot 121 that extends throughout a length of the inner screw 120 and that may be complementary to the one or more protrusion 151. In some embodiments, the one or more protrusion 151 is sized to engage the one the more slot 121, such that the inner screw 120 may advance through the opening 155 without rotating due to resistance between the one or more protrusion 151 and a corresponding one or more slot 121. In some embodiments, the ARS 150 comprises a one or more tail 152 that extend from a surface of the base. For example, the one or more tail 152 extend from a proximal side of the base 154 toward the screw support 160. In some embodiments, the one or more tail 152 extend along an axis that is perpendicular to the base 154 of the ARS 150. In some embodiments, the screw support 160 comprises a one or more aperture 162. The one or more aperture 162 may be aligned with and sized to receive a corresponding one or more tail 152.

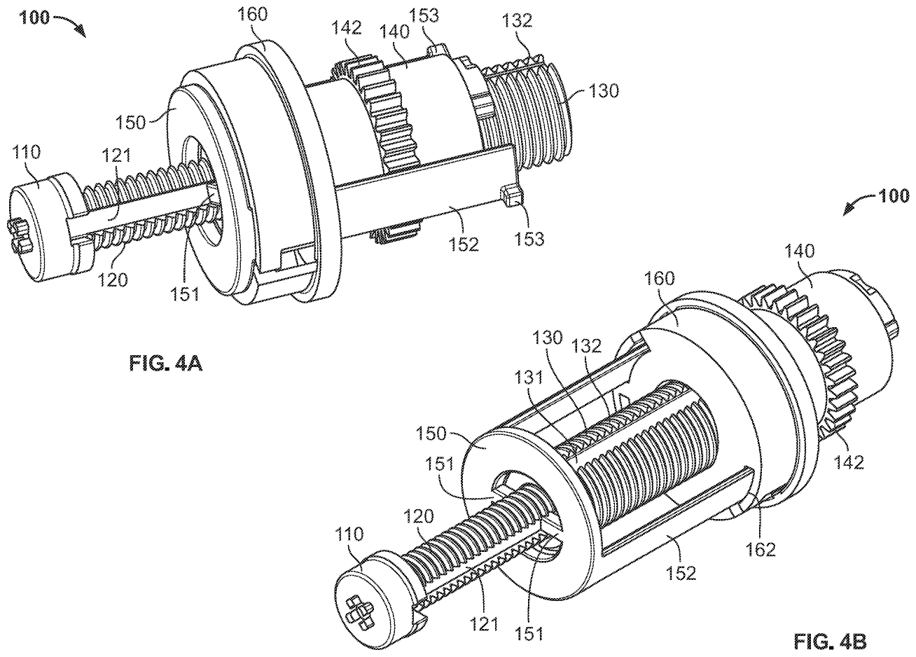

FIGS. 4A-4B illustrate perspective views of the plunger driver 100 in a partially extended state and a fully extended state, respectively. In some embodiments, the ARS 150 accomplishes anti-rotation control to enable extension of the pusher 110 into the reservoir 22. In some embodiments, the one or more protrusion 151 is configured to engage the one or more slot 121 of inner screw 120 to transfer any torque applied to inner screw 120 to ARS 150. In some embodiments, the one or more tail 152 is inserted into the one or more aperture 162 to engage the one or more aperture 162. These engagements prevent rotation of the ARS 150 with respect to the screw support 160, and the inner screw 120 cannot rotate with respect to the ARS 150. Therefore, as the outer screw 130 is rotated, the inner screw 120 advances into the reservoir 22 away from the screw support 160 and without rotating relative to the screw support 160, thereby translating rotational movement of the outer screw 130 into linear movement of the inner screw 120. In some embodiments, the outer screw 130 is driven to rotate by the drive nut 140, based on the engagement between the one or more inner protrusion 141 and the one or more outer slot 131. In some embodiments, the drive nut 140 may be driven via the motor and the gear train. In some embodiments, the pusher 110, which is anchored between the inner screw 120 and the plunger 28. advances into the reservoir 22 based on the linear movement of the inner screw 120. In particular, this is a result of torque transfer from the drive nut 140 to inner screw 120 via the outer screw 130, which in turn transfers the applied torque to the ARS 150 to prevent rotation of inner screw 120.

The one or more tail 152 may allow the outer screw 130 to be advanced relative to the screw support 160. In particular, the length of the one or more tail 152 may correspond to a desired length of extension of the outer screw 130 from the drive nut 140. As shown in FIGS. 4A and 4B, as the outer screw is advanced from the screw support, the outer screw also advances the ARS away from the screw support, and the distance of such advancement may be controlled by varying the length of the one or more tail 152. In some embodiments, one or more tab 153 is positioned at a distal end of a corresponding one or more tail 152 in a desired position corresponding to the desired length of extension of the outer screw 130 from the drive nut 140.

The screws 120 and 130 have right-handed threads, for example, but could also be designed to all have left-handed threads. For example, each of the screws 120 and 130 has the same inner and/or outer screw designs with same pitch and slight variations on the other parameters. In some embodiments, the plunger driver 100 comprises three nested screws (not shown). In some embodiments, each of the three nested screws has the same handedness of threads. In some embodiments, the plunger driver 100 comprises two or more nested screws (not shown). In some embodiments, each of the two or more nested screws has the same handedness of threads. In some embodiments, each of the screws 120 and 130 or two or more nested screws extends sequentially based on torque values of the screw, e.g., lowest first, highest last.

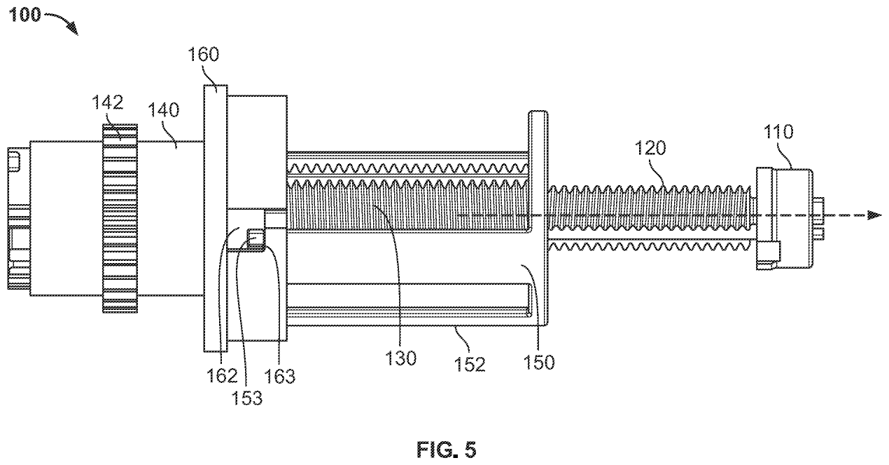

In accordance with an example embodiment, a length of ARS 150 is dimensioned such that, when the screws are all nested or collapsed, they are all contained in the ARS 150. In some embodiments, the one or more tail 152 is a desired length. The desired length corresponds to a potential extension length of the pusher 110 relative to the drive nut 140. As illustrated in FIG. 5, the one or more tab 153 on the one or more tail 152 engages a detent 163 within each of the one or more aperture 162. The one or more tab 153 may function as a stopper to prevent disassembly of the ARS 150 from screw support 160. Therefore, the one or more tab 153 serves to limit an extension length of the pusher 110 from the drive nut 140.

In some embodiments, the fluid delivery assembly 10 may be disposable. In some embodiments, the reservoir 22 may contain medication supply to be administered to a patient. In some embodiments, the pusher optionally comprises components that allow for venting. In some embodiments, the pusher comprises at least one through hole for venting. In some embodiments, the pusher comprises indents along at least a portion of its perimeter for venting. The venting features can facilitate a higher rate of delivery by providing a more open flow path.

It is to be understood that the motor can control the plunger driver 100 to move the telescoping screw members incrementally from the fully retracted to the fully extended positions shown to deliver respective designated dose amounts of fluid from a fluid chamber portion 64 of the reservoir 22. The motor and gear train rotate a drive nut 140 on the plunger driver 100. The gear train can have different configurations. For example, the gear train can also be in the form of a ratchet indexing mechanism or other indexing mechanism that precisely rotates the drive nut 140 by a mechanically controlled amount. The motor and related gear train components and the drive nut 140 of the plunger driver 100 can be mounted with respect to each other.

In some embodiments, the fluid delivery assembly 10 further comprises a thrust bearing feature provided relative to the plunger driver and a support structure of the plunger driver to minimize axial thrust load from the inner screw and the outer screw. In some embodiments, the thrust bearing feature comprises a cap disposed on the drive nut, the cap having a boss for contacting a portion of the support structure to resist an axial thrust load directed toward the proximal end of the plunger driver and generated by the plunger driver, plunger, or fluid in the fluid chamber. In some embodiments, the fluid delivery assembly 10 further comprises an encoder provided relative to the plunger driver to generate feedback data related to operation of the plunger driver. In some embodiments, a distal end of the inner screw is heat staked at a distal side of the pusher at a through-hole.

In some embodiments, the plunger 28 may comprise a stopper assembly (not shown) to prevent leakage of any fluid retained in a fluid chamber portion 64 of the reservoir 22. In some embodiments, the stopper assembly can comprise, for example, an elastic member comprising elastic material similar to a syringe stopper and configured as disc mounted to a surface of a plunger 28 disc or as a band of material surrounding the plunger 28 disc. Alternatively, the plunger 28 can be configured to have one or more (e.g., two) circumferential groove dimensioned to accommodate respective O-ring(s). For example, using two O-rings increases stability (e.g., even in spite of an increase in length). Depending on dose accuracy requirements, a single O-ring can be a viable option; however, for high precision, two O-rings are particularly beneficial.

The configuration of the plunger driver 100 components with respect to the reservoir 22 and the plunger 28 realizes a number of advantages. For example, having a plunger driver 100 mounted at a proximal end of the reservoir 22 and having a nested configuration that does not extend into the reservoir until the outermost drive screw is rotated optimizes use of the reservoir chamber for fluid delivery instead of having to accommodate pre-delivery plunger driver components. In addition, the overall length of the reservoir can be substantially the same as the length of a housing, with the addition of a small amount of headspace to accommodate the gear train connection to the outer teeth 142 of the drive nut 140. Thus, the overall footprint of the pump mechanism is minimized as well as the longitudinal axis dimension of the fluid delivery device housing. The use of the plunger 28 and plunger driver 100 design also minimizes contact of the pump mechanism with the fluid being delivered to ensure biocompatibility between the fluid and the fluid delivery housing. The example embodiments described herein employ nested telescoping screws and ARS of appropriate size and thread configuration to achieve a controlled movement of a syringe-barrel-type reservoir plunger 28. Screw-thread technology is well-defined and understood, and is capable of repeatable, powerful motion. When driven with an appropriate resolution-controlled motion by the motor, the nested screws 120 and 130 can provide accurate movement under virtually all environmental conditions. Further, the drive mechanism (e.g., the plunger driver 100) the does not affect the basic volume of the fluid chamber 64 where the drug resides, thus having no impact on any compatibility issues.

In some embodiments, the fluid delivery assembly 10 may be implemented in a variety of fluid delivery devices. Exemplary types of such fluid delivery devices may include, but are not limited to, pump systems, medication pens or similar infusion or injection devices for delivering fluids to a patient, such as, for example, the pump or medication pen systems of co-pending U.S. Patent Application Nos. 63/591,363 and 63/507,657, incorporated by reference in their entirety. In some embodiments, the fluid delivery assembly 10 may be implemented in a portable pump system, such as, for example, skin-based patch pumps, belt-based pumps, or any other wearable pump system. In some embodiments, the fluid delivery device may be any device for delivering medicament, such as insulin, or other pharmaceutical or therapeutic drug to a user. In some embodiments, the fluid delivery assembly 10 may be built into a housing of the fluid delivery device or may be a medicament cartridge that may be removably added to the fluid delivery device. In some embodiments, the fluid delivery assembly 10 may be incorporated in its entirety, or by implementing individual components selected from the reservoir 22, the plunger 28, or the plunger driver 100. In some embodiments, the reservoir 22 can be similar in materials to a syringe barrel and associated stopper. In some embodiments, the fluid delivery device may be controlled remotely by a remote control (RC) device. In some embodiments, the RC device is wireless or wired. In some embodiments, the wireless RC device may be a mobile phone, dedicated wireless remote controller, or other control device. In some embodiments, the fluid delivery device may be coupled to a user interface.

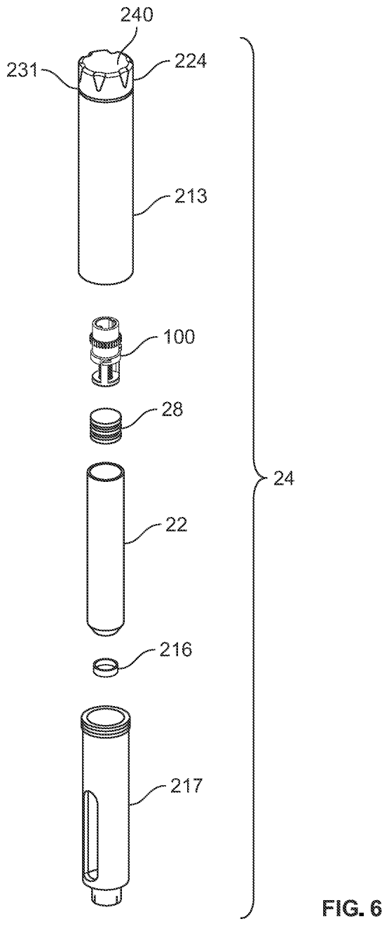

Referring to FIG. 6, the fluid delivery assembly 10 is depicted as implemented within a medication delivery pen 24, according to some embodiments. In some embodiments, the delivery pen 24 may be disposable. In some embodiments, the delivery pen 24 may include an outer sleeve 213 and a lower housing 217. In some embodiments, the outer sleeve 213 may provide a gripping surface for a user to grip when administering medication to a patient. In some embodiments, the delivery pen 24 includes a dose knob 224 positioned at a proximal end 231 of the outer sleeve 213. The dose knob 224 may be rotatable with respect to the outer sleeve 213 of the delivery pen 24. In some embodiments, the user may rotate the dose knob 224 to selectively set a desired volume of a dose of medication to be injected to a patient. In some embodiments, rotation of the dose knob 224 in a first direction may axially translate the dose knob 224 away from the proximal end 231 of the outer sleeve 213, thereby increasing the volume of a dose of medication to be administered with the delivery pen 24. In some embodiments, rotation of the dose knob 224 in a second direction may axially translate the dose knob 224 toward the proximal end 231 of the outer sleeve 213, thereby decreasing the volume of a dose of medication to be administered with the delivery pen 24. In some embodiments, a button 240 may be coupled to a proximal end of the dose knob 224. A user may apply an axial force to the button 240 to depress the button 240 and dose knob 224 axially toward the proximal end 231 of the outer sleeve 213, thereby activating the plunger driver 100 to administer a dose of medication from the delivery pen 24.

In some embodiments, at least some of the fluid delivery assembly 10 may be disposed within the outer sleeve 213, the lower housing 217, or a combination thereof. In some embodiments, the outer sleeve 213 may receive the fluid delivery assembly 10 in its entirety. In some embodiments, the outer sleeve 213 may receive the plunger driver 100. In some embodiments, the outer sleeve 213 may contain reservoir 22. In some embodiments, the lower housing 217 may contain the fluid delivery assembly 10 in its entirety. In some embodiments, the lower housing 217 may contain the plunger driver 100. In some embodiments, the lower housing 217 may contain the reservoir 22. Generally, the button 240 and dose knob 224 are understood to be coupled to the fluid delivery assembly 10, such that depression of the button 240 and dose knob 224 in the distal direction injects the dosed medication via the plunger driver 100 and a plunger 28 through the reservoir 22. The distal movement of the plunger 28 within the reservoir 22 causes medication to be forced into a needle (not shown) of a pen needle (not shown). The reservoir 22 may be sealed by a septum 216, which may be punctured by the needle located within the pen needle. In some embodiments, the pen needle may be screwed onto the lower housing 217, although other attachment means may be used. It should be appreciated that the foregoing description is merely one representative example of the delivery pen 24, and other designs for the delivery pen 24 are contemplated herein.

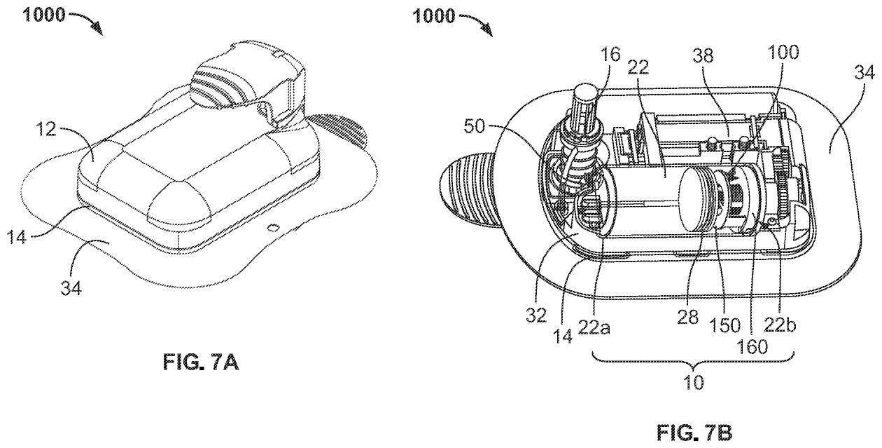

Referring to FIGS. 7A-7B, a perspective view of a wearable pump system 1000 is depicted, with and without a top enclosure, respectively, in accordance with an example embodiment in which a fluid delivery assembly 10 of the present disclosure may be incorporated. In some embodiments, the wearable pump system 1000 is an insulin patch pump. Referring to FIG. 7A, the wearable pump system 1000 is shown to comprise a top enclosure 12, a bottom enclosure 14, and an adhesive patch 34.

Referring to FIG. 7B, the wearable pump system 1000, with the top enclosure 12 removed, comprises a fluid delivery assembly 10. The wearable pump system 1000 is further shown to comprise an insertion mechanism 16, a reservoir 22, a power supply 38, and a plunger driver 100 of the present disclosure. The plunger driver 100 is shown connected to the reservoir 22 at the proximal end or capped end 22b of the reservoir 22, driving a plunger 28 that couples to a pusher 110 on the plunger driver 100. In some embodiments, the screw support 160 is inserted into the proximal end of the reservoir to support the plunger driver 100 within the reservoir. In some embodiments, the reservoir 22, plunger driver 100, and power supply 38 may be constrained within the housing 14 of the wearable pump system 1000 by an internal bracket 32 and the housing 14 to minimize relative motion of the components during device operation. In some embodiments, the plunger driver 100 is replaceable. In some embodiments, the wearable pump system 1000 includes at least a processor and a non-transitory computer-readable storage medium storing one or more programs configured for execution by the processor. The processor may be configured to control various components of the wearable pump system 1000, such as, for example, to adjust a fluid delivery rate via the fluid delivery assembly 10, transmit or receive information by communicating with the RC device, activate the insertion mechanism 16, manage power, etc. In some embodiments, the insertion mechanism 16 is user-accessible and user-actuatable. In some embodiments, the insertion mechanism 16, upon activation, inserts a catheter into the user's skin to establish a fluid path from a fluid reservoir to the user via the catheter and other components in the fluid path. In some embodiments, the reservoir 22 can be prefilled and the plunger driver 100 initially situated in a retracted position. Alternatively, the fluid delivery device can be provided with a fill port and fluid path from the fill port to the reservoir 22. The fill port can be configured for filling by a user with a syringe, or by using a filling station that fluidically couples to the fill port.

The technical solution of the example embodiments is based on a basic screw-drive mechanism where lifting torque is a function of applied axial load (force or pressure), thread pitch, friction parameters, and diameter. In some cases, the equations may be further expanded to capture the full details of thread geometry such as flank and lead angle, and many other special parameters. Industry standard sizes for ACME threads can generally be used to adjust the balance of lifting torque, power required, efficiency, and other functional parameters such as smoothness of operation and cost. Other thread forms can also be used, such as Buttress threads, to accurately control load-transfer, and minimize dosing errors. Each screw design may affect torque; therefore, changes should be made in a manner that is congruent with the capabilities of the motor and gearbox or index drive sub-system.

The design employed by the example embodiments lends itself to be driven with gear-reduction transmissions at very small scales. The torque required to move a gear is independent of the number of gears used in the system, and is mostly affected by material and geometry choices for the threads. Small motors and low gearbox ratios can therefore be employed, thereby yielding a compact device 10. Conversely, the torque will be different on each screw, with the smaller torque being on the innermost screw and the largest torque being on the outermost screw. The efficiency of power transmission is affected by the many interfaces, which can reduce overall efficiency but which can be adjusted to an acceptable level using adjusted parameters for the equations to determine desired lifting torque. Regardless, if battery power, or any other input power, is abundantly available, this design has the potential to create highly accurate pumps for many drug therapies, unlike any type of medical drug delivery pump currently available.

### Exemplary Embodiments

Example embodiments of the present disclosure realize several advantages such as minimizing the device size envelope or form factor, while retaining the beneficial features of highly reliable and proven systems such as medication pens and pen needles, syringes, or more expensive, non-portable pumping systems that employ a lead screw drive mechanism, such as the plunger driver 100 described herein. In accordance with example embodiments described herein, a nesting telescopic screw design is employed that enables the use of syringe-based drug containers or similar reservoirs, which have been proven to be drug-friendly or biocompatible with drugs and other fluids delivered via fluid delivery devices. Example embodiments of the present disclosure prevents rotation of a plunger in circular fluid delivery devices, while retaining the features of highly reliable and proven systems such as medication pens and pen needles, syringes, or more expensive, non-portable pumping systems that employ a lead screw drive mechanism.

In some aspects, the techniques described herein relate to a fluid delivery system, including: a reservoir including an outlet port at a distal end, and plunger movable along a longitudinal axis of the reservoir, the plunger configured to provide a seal with respect to inner walls of the reservoir to prevent fluid provided in a fluid chamber defined on a first side of the plunger and preventing the outlet port from leaking into a portion of the reservoir defined by a second side of the plunger; and a plunger driver, the plunger driver including: a pusher mounted at a proximal end of an inner screw, wherein the pusher engages the plunger; an outer screw concentrically surrounding the inner screw, wherein an inner surface of the outer screw includes first threads, an outer surface of the inner screw includes second threads, and the first threads engage the second threads; a drive nut concentrically surrounding and engaging the outer screw; and an anti-rotation sleeve (ARS) engaged with a screw support and the inner screw, such that, when the drive nut is rotated, the pusher moves the plunger without rotating from a nested configuration that does not extend into the reservoir to an extended configuration that extends from a proximal end of the reservoir into the reservoir.

In some aspects, the reservoir is a syringe barrel-type container.

In some aspects, the pusher is disposed between the plunger and a distal end of the inner screw, the pusher abutting a proximal side of the plunger and configured to move along the longitudinal axis of the reservoir in response to rotation of the outer screw.

In some aspects, an inner surface of the outer screw includes a first mating surface, an outer surface of the inner screw includes a second mating surface, and the first mating surface engages the second mating surface, such that at least one of the first mating surface and second mating surface is configured to relatively rotate.

In some aspects, at least one of the inner screw and the outer screw is configured to relatively rotate, wherein the second mating surface is configured to cooperate with the first mating surface to advance the inner screw within the outer screw when the outer screw is rotated.

In some aspects, the drive nut includes a one or more inner protrusion, the outer screw includes a one or more outer slot, and the one or more inner protrusion engages the one or more outer slot to achieve engagement between the drive nut and outer screw.

In some aspects, the drive nut further includes outer teeth, wherein the outer teeth are configured to engage a motor.

In some aspects, the outer teeth are configured to directly engage the motor.

In some aspects, the outer teeth are configured to indirectly engage the motor through at least one of a gear train and an outermost drive screw.

In some aspects, the outer screw includes an outer surface threading and the screw support includes an inner surface threading, wherein the outer surface threading engages the inner surface threading such that torque transfer is prevented between the outer screw and screw support and that the outer screw may advance through the screw support.

In some aspects, the inner screw includes a one or more slot that extends throughout a length of the inner screw and the ARS includes a one or more protrusion, wherein the one or more slot engages the one or more protrusion such that torque applied to the inner screw is transferred to the ARS.

In some aspects, the ARS includes a one or more tail, wherein each of the one or more tail includes a tab is positioned at a distal end of a corresponding one or more tail in a desired position corresponding to a desired length of extension of the pusher from the drive nut.

In some aspects, the screw support includes a one or more aperture, wherein the one or more tail is inserted into the one or more aperture to engage the ARS such that the ARS is prevented from rotating with respect to the screw support.

In some aspects, the tab on the one or more tail engages a detent within each of the one or more aperture such that the ARS is prevented from disassembling from the screw support.

In some aspects, a length of ARS is dimensioned such that, when the inner screw and the outer screw are nested or collapsed, the inner screw and the outer screw are all contained in the ARS.

In some aspects, the techniques described herein relate to an anti-rotation device, including: a pusher mounted at a proximal end of an inner screw, wherein the pusher engages a plunger; an outer screw concentrically surrounding the inner screw, wherein an inner surface of the outer screw includes first threads, an outer surface of the inner screw has second threads, and the first threads engage the second threads; a drive nut concentrically surrounding and engaging the outer screw; and an anti-rotation sleeve (ARS) engaged with a screw support and the inner screw, such that, when the drive nut is rotated, the pusher moves without rotating from a nested configuration to an extended configuration.

In some aspects, the pusher is disposed between the plunger and a distal end of the inner screw, the pusher abutting a proximal side of the plunger and configured to move along a longitudinal axis in response to rotation of the outer screw.

In some aspects, an inner surface of the outer screw includes a first mating surface, an outer surface of the inner screw includes a second mating surface, and the first mating surface engages the second mating surface, such that at least one of the first mating surface and second mating surface is configured to respectively rotate.

In some aspects, at least one of the inner screw and the outer screw is configured to relatively rotate, wherein the second mating surface is configured to cooperate with the first mating surface to advance the inner screw within the outer screw when the outer screw is rotated.

In some aspects, the drive nut includes a one or more inner protrusion, the outer screw includes a one or more outer slot, and the one or more inner protrusion engages the one or more outer slot to achieve engagement between the drive nut and outer screw.

In some aspects, the drive nut further includes outer teeth, wherein the outer teeth are configured to engage a motor.

In some aspects, the outer teeth are configured to directly engage the motor.

In some aspects, the outer teeth are configured to indirectly engage the motor through at least one of a gear train and an outermost drive screw.

In some aspects, the outer screw includes an outer surface threading and the screw support includes an inner surface threading, wherein the outer surface threading engages the inner surface threading such that torque transfer is prevented between the outer screw and screw support and that the outer screw may advance through the screw support.

In some aspects, the inner screw includes a one or more slot that extends throughout a length of the inner screw and the ARS includes a one or more protrusion, wherein the one or more slot engages the one or more protrusion such that torque applied to the inner screw is transferred to the ARS.

In some aspects, the ARS includes a one or more tail, wherein each of the one or more tail includes a tab is positioned at a distal end of a corresponding one or more tail in a desired position corresponding to a desired length of extension of the pusher from the drive nut.

In some aspects, the screw support includes a one or more aperture, wherein the one or more tail is inserted into the one or more aperture to engage the ARS such that the ARS is prevented from rotating with respect to the screw support.

In some aspects, the tab on the one or more tail engages a detent within each of the one or more aperture such that the ARS is prevented from disassembling from the screw support.

In some aspects, a length of ARS is dimensioned such that, when the inner screw and the outer screw are nested or collapsed, the inner screw and the outer screw are all contained in the ARS.

In some aspects, the techniques described herein relate to a fluid delivery device, the fluid delivery device including a fluid delivery system or an anti-rotation device described herein.

Additional non-limiting embodiments of the present disclosure are set out in the following clauses:
Clause 1. A fluid delivery system, comprising: an inner screw having a pusher disposed at a distal end of the inner screw to engage and linearly translate a plunger within a reservoir to dispense a medication from the reservoir; an outer screw concentrically surrounding the inner screw, wherein an inner surface of the outer screw engages with an outer surface of the inner screw such that a rotational movement of the outer screw advances the inner screw from a nested configuration to an extended configuration; a screw support configured to engage with the outer screw; and an anti-rotation sleeve (ARS) engaged with the screw support and the inner screw, such that, when the outer screw is rotated, the inner screw is advanced without rotating.

Clause 2. The fluid delivery system of clause 1, wherein the ARS comprises: a base with an opening sized to receive the inner screw; one or more protrusion extending inwardly into the opening; and one or more tail extending along an axis perpendicular to the base.

Clause 3. The fluid delivery system of clause 1 or clause 2, wherein the inner screw further comprises one or more slot that are sized to receive the one or more protrusion of the ARS to prevent rotation of the inner screw as the inner screw is advanced.

Clause 4. The fluid delivery system of any one of clauses 1-3, wherein the one or more slot extends throughout a length of the inner screw.

Clause 5. The fluid delivery system of any one of clauses 1-4, wherein the screw support comprises one or more aperture aligned with and sized to receive the one or more tail, and wherein the one or more tail is insertable into the one or more aperture such that the ARS is prevented from rotating with respect to the screw support.

Clause 6. The fluid delivery system of any one of clauses 1-5, wherein the screw support further comprises an opening sized to receive the outer screw, wherein an inner surface of the opening engages an outer surface of the outer screw such that rotation of the outer screw advances the outer screw through the opening.

Clause 7. The fluid delivery system of any one of clauses 1-6, wherein each of the one or more tail comprises a tab positioned at a distal end of a corresponding one or more tail in a desired position corresponding to a desired length of extension of the outer screw from the screw support, and wherein the tab on the one or more tail is engageable with a detent within each of the one or more aperture such that the ARS is prevented from disengaging from the screw support.

Clause 8. The fluid delivery system of any one of clauses 1-7, further comprising a drive nut comprising: one or more inner protrusion along an inner surface of the drive nut; and outer teeth along an outer surface of the drive nut, wherein the outer screw comprises one or more outer slot, and the one or more inner protrusion are sized to engage the one or more outer slot, and wherein the outer teeth are configured to engage a motor such that the motor drives rotation of the drive nut.

Clause 9. The fluid delivery system of any one of clauses 1-8, wherein the outer teeth are configured to engage the motor directly or indirectly.

Clause 10. The fluid delivery system of any one of clauses 1-9, wherein the inner surface of the outer screw is configured to cooperate with the outer surface of the inner screw to advance the inner screw within the outer screw when the outer screw is rotated.

Clause 11. The fluid delivery system of any one of clauses 1-10, wherein the reservoir is circular in shape.

Clause 12. A fluid delivery system, comprising: a reservoir comprising an outlet port at a distal end, and plunger movable along a longitudinal axis of the reservoir, the plunger configured to provide a seal with respect to inner walls of the reservoir; and a plunger driver, the plunger driver comprising: an inner screw being configured to advance the plunger along the longitudinal axis of the reservoir; an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir; a screw support configured to be inserted into the reservoir at a proximal end of the reservoir and configured to engage with the outer screw; and an anti-rotation sleeve (ARS) engaged with the screw support and the inner screw, such that, when the outer screw is rotated, the inner screw is advanced without rotating.

Clause 13. The fluid delivery system of clause 11 or clause 12, wherein the ARS comprises: a base with an opening sized to receive the inner screw; one or more protrusion extending inwardly into the opening; and one or more tail extending along an axis perpendicular to the base.

Clause 14. The fluid delivery system of any one of clauses 11-13, wherein the inner screw further comprises one or more slot that are sized to receive the one or more protrusion of the ARS to prevent rotation of the inner screw as the inner screw is advanced.

Clause 15. The fluid delivery system of any one of clauses 11-14, wherein the one or more slot extends throughout a length of the inner screw.

Clause 16. The fluid delivery system of any one of clauses 11-15, wherein the screw support comprises one or more aperture aligned with and sized to receive the one or more tail, and wherein the one or more tail is insertable into the one or more aperture such that the ARS is prevented from rotating with respect to the screw support.

Clause 17. The fluid delivery system of any one of clauses 11-16, wherein the screw support further comprises an opening sized to receive the outer screw, wherein an inner surface of the opening engages an outer surface of the outer screw such that rotation of the outer screw advances the outer screw through the opening.

Clause 18. The fluid delivery system of any one of clauses 11-17, wherein each of the one or more tail comprises a tab positioned at a distal end of a corresponding one or more tail in a desired position corresponding to a desired length of extension of the outer screw from the screw support, and wherein the tab on the one or more tail is engageable with a detent within each of the one or more aperture such that the ARS is prevented from disengaging from the screw support.

Clause 19. The fluid delivery system of any one of clauses 11-18, wherein the plunger driver further comprises a drive nut comprising: one or more inner protrusion along an inner surface of the drive nut; and outer teeth along an outer surface of the drive nut, wherein the outer screw comprises one or more outer slot, and the one or more inner protrusion are sized to engage the one or more outer slot, and wherein the outer teeth are configured to engage a motor such that the motor drives rotation of the drive nut.

Clause 20. The fluid delivery system of any one of clauses 11-19, wherein the outer teeth are configured to engage the motor directly or indirectly.

Clause 21. The fluid delivery system of any one of clauses 11-20, wherein the plunger driver further comprises a pusher disposed at a distal end of the inner screw to engage and linearly translate the plunger as the inner screw advances through the reservoir.

Clause 22. The fluid delivery system of any one of clauses 11-21, wherein the inner surface of the outer screw is configured to cooperate with the outer surface of the inner screw to advance the inner screw within the outer screw when the outer screw is rotated.

Clause 23. The fluid delivery system of any one of clauses 11-22, wherein the reservoir is circular in shape.

Clause 24. A fluid delivery device comprising a fluid delivery system of any one of clauses 1-23.

Clause 25. A fluid delivery device, the fluid delivery device comprising: a housing; a reservoir disposed within the housing, the reservoir comprising an outlet port at a distal end, and plunger movable along a longitudinal axis of the reservoir, the plunger configured to provide a seal with respect to inner walls of the reservoir; a plunger driver, the plunger driver comprising: an inner screw being configured to advance the plunger along the longitudinal axis of the reservoir; an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir; a screw support configured to be inserted into the reservoir at a proximal end of the reservoir and configured to engage with the outer screw; and an anti-rotation sleeve (ARS) engaged with the screw support and the inner screw, such that, when the outer screw is rotated, the inner screw is advanced without rotating; a drive nut concentrically surrounding and engaging the outer screw, wherein the drive nut is configured to be driven by an actuator to rotate the outer screw; and a fluid path comprising a proximal end in fluid communication with the outlet port of the reservoir and a distal end configured to be inserted into a patient.

Clause 26. The fluid delivery device of clause 25, wherein the fluid delivery device comprises a patch pump.

Clause 27. The fluid delivery device of clause 25 or clause 26, wherein the fluid delivery device comprises a medication pen.

Clause 28. The fluid delivery device of any one of clauses 25-27, wherein the actuator comprises a motor.

Clause 29. The fluid delivery device of any one of clauses 25-28, wherein the ARS comprises: a base with an opening sized to receive the inner screw; one or more protrusion extending inwardly into the opening; and one or more tail extending along an axis perpendicular to the base.

Clause 30. The fluid delivery device of any one of clauses 25-29, wherein the inner screw further comprises one or more slot that are sized to receive the one or more protrusion of the ARS to prevent rotation of the inner screw as the inner screw is advanced.

Clause 31. The fluid delivery device of any one of clauses 25-30, wherein the one or more slot extends throughout a length of the inner screw.

Clause 32. The fluid delivery device of any one of clauses 25-31, wherein the screw support comprises one or more aperture aligned with and sized to receive the one or more tail, and wherein the one or more tail is insertable into the one or more aperture such that the ARS is prevented from rotating with respect to the screw support.

Clause 33. The fluid delivery device of any one of clauses 25-32, wherein the screw support further comprises an opening sized to receive the outer screw, wherein an inner surface of the opening engages an outer surface of the outer screw such that rotation of the outer screw advances the outer screw through the opening.

Clause 34. The fluid delivery device of any one of clauses 25-33, wherein each of the one or more tail comprises a tab positioned at a distal end of a corresponding one or more tail in a desired position corresponding to a desired length of extension of the outer screw from the screw support, and wherein the tab on the one or more tail is engageable with a detent within each of the one or more aperture such that the ARS is prevented from disengaging from the screw support.

Clause 35. The fluid delivery device of any one of clauses 25-34, further comprising a drive nut comprising: one or more inner protrusion along an inner surface of the drive nut; and outer teeth along an outer surface of the drive nut, wherein the outer screw comprises one or more outer slot, and the one or more inner protrusion are sized to engage the one or more outer slot, and wherein the outer teeth are configured to engage a motor such that the motor drives rotation of the drive nut.

Clause 36. The fluid delivery device of any one of clauses 25-35, wherein the outer teeth are configured to engage the motor directly or indirectly.

Clause 37. The fluid delivery device of any one of clauses 25-36, further comprising a pusher disposed at a distal end of the inner screw to engage and linearly translate as the inner screw advances through the outer screw.

Clause 38. The fluid delivery device of any one of clauses 25-37, wherein the inner surface of the outer screw is configured to cooperate with the outer surface of the inner screw to advance the inner screw within the outer screw when the outer screw is rotated.

As utilized herein, the terms “comprise” and “comprising” are intended to be construed as being inclusive, not exclusive. As utilized herein, the terms “exemplary,” “example.” and “illustrative,” are intended to mean “serving as an example, instance, or illustration,” and should not be construed as indicating, or not indicating, a preferred or advantageous configuration relative to other configurations. As utilized herein, the terms “about.” “generally,” and “approximately” are intended to cover variations that may exist in the upper and lower limits of the ranges of subjective or objective values, such as variations in properties, parameters, sizes, and dimensions. In one non-limiting example, the terms “about,” “generally,” and “approximately” mean at, or plus 10 percent or less, or minus 10 percent or less. In one non-limiting example, the terms “about,” “generally,” and “approximately” mean sufficiently close to be deemed by one of skill in the art in the relevant field to be included. As utilized herein, “distal” refers to the direction toward or nearer a patient end of a delivery pump (i.e., a needle for injection) or other device. As utilized herein, “proximal” refers to the direction toward or nearer a user end of the delivery pump (i.e., a user button for actuation) or other device. As utilized herein, “axial” refers to the longitudinal direction of the of the delivery pump or other device. As utilized herein, “radial” refers to the direction perpendicular to the axial direction. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by the appended claims and the applicable rules of law.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed. It is intended that the following claims define the scope of the disclosure and that methods and structures within the scope of these claims and their equivalents be covered thereby. It is also to be understood that the following claims are to cover all generic and specific features of the disclosure described herein, and all statements of the scope of the disclosure which, as a matter of language, might be said to fall therebetween,

All patents and publications mentioned in this specification are herein incorporated by reference to the same extent as if each independent patent and publication was specifically and individually indicated to be incorporated by reference.

1. A fluid delivery system, comprising:
an inner screw having a pusher disposed at a distal end of the inner screw to engage and linearly translate a plunger within a reservoir to dispense a medication from the reservoir;
an outer screw concentrically surrounding the inner screw, wherein an inner surface of the outer screw engages with an outer surface of the inner screw such that a rotational movement of the outer screw advances the inner screw from a nested configuration to an extended configuration;
a screw support configured to engage with the outer screw; and
an anti-rotation sleeve (ARS) engaged with the screw support and the inner screw, such that, when the outer screw is rotated, the inner screw is advanced without rotating.

2. The fluid delivery system of claim 1, wherein the ARS comprises:
a base with an opening sized to receive the inner screw;
one or more protrusion extending inwardly into the opening; and
one or more tail extending along an axis perpendicular to the base.

3. The fluid delivery system of claim 2, wherein the inner screw further comprises one or more slot that are sized to receive the one or more protrusion of the ARS to prevent rotation of the inner screw as the inner screw is advanced.

4. The fluid delivery system of claim 3, wherein the one or more slot extends throughout a length of the inner screw.

5. The fluid delivery system of claim 2, wherein the screw support comprises one or more aperture aligned with and sized to receive the one or more tail, and wherein the one or more tail is insertable into the one or more aperture such that the ARS is prevented from rotating with respect to the screw support.

6. The fluid delivery system of claim 5, wherein the screw support further comprises an opening sized to receive the outer screw, wherein an inner surface of the opening engages an outer surface of the outer screw such that rotation of the outer screw advances the outer screw through the opening.

7. The fluid delivery system of claim 5, wherein each of the one or more tail comprises a tab positioned at a distal end of a corresponding one or more tail in a desired position corresponding to a desired length of extension of the outer screw from the screw support, and wherein the tab on the one or more tail is engageable with a detent within each of the one or more aperture such that the ARS is prevented from disengaging from the screw support.

8. The fluid delivery system of claim 1, further comprising a drive nut comprising:
one or more inner protrusion along an inner surface of the drive nut; and
outer teeth along an outer surface of the drive nut,
wherein the outer screw comprises one or more outer slot, and the one or more inner protrusion are sized to engage the one or more outer slot, and
wherein the outer teeth are configured to engage a motor such that the motor drives rotation of the drive nut.

9. The fluid delivery system of claim 8, wherein the outer teeth are configured to engage the motor directly or indirectly.

10. The fluid delivery system of claim 1, wherein the inner surface of the outer screw is configured to cooperate with the outer surface of the inner screw to advance the inner screw within the outer screw when the outer screw is rotated.

11. The fluid delivery system of claim 1, wherein the reservoir is circular in shape.

12. A fluid delivery system, comprising:
a reservoir comprising an outlet port at a distal end, and plunger movable along a longitudinal axis of the reservoir, the plunger configured to provide a seal with respect to inner walls of the reservoir; and
a plunger driver, the plunger driver comprising:
an inner screw being configured to advance the plunger along the longitudinal axis of the reservoir;
an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir;
a screw support configured to be inserted into the reservoir at a proximal end of the reservoir and configured to engage with the outer screw; and
an anti-rotation sleeve (ARS) engaged with the screw support and the inner screw, such that, when the outer screw is rotated, the inner screw is advanced without rotating.

13. The fluid delivery system of claim 12, wherein the ARS comprises:
a base with an opening sized to receive the inner screw;
one or more protrusion extending inwardly into the opening; and
one or more tail extending along an axis perpendicular to the base.

14. The fluid delivery system of claim 13, wherein the inner screw further comprises one or more slot that are sized to receive the one or more protrusion of the ARS to prevent rotation of the inner screw as the inner screw is advanced, wherein the one or more slot extends throughout a length of the inner screw.

15. (canceled)

16. The fluid delivery system of claim 13, wherein the screw support comprises one or more aperture aligned with and sized to receive the one or more tail, wherein the one or more tail is insertable into the one or more aperture such that the ARS is prevented from rotating with respect to the screw support, wherein each of the one or more tail comprises a tab positioned at a distal end of a corresponding one or more tail in a desired position corresponding to a desired length of extension of the outer screw from the screw support, and wherein the tab on the one or more tail is engageable with a detent within each of the one or more aperture such that the ARS is prevented from disengaging from the screw support.

17. (canceled)

18. (canceled)

19. The fluid delivery system of claim 12, wherein the plunger driver further comprises a drive nut comprising:
one or more inner protrusion along an inner surface of the drive nut; and
outer teeth along an outer surface of the drive nut,
wherein the outer screw comprises one or more outer slot, and the one or more inner protrusion are sized to engage the one or more outer slot, and
wherein the outer teeth are configured to engage a motor such that the motor drives rotation of the drive nut.

20. (canceled)

21. The fluid delivery system of claim 12, wherein the plunger driver further comprises a pusher disposed at a distal end of the inner screw to engage and linearly translate the plunger as the inner screw advances through the reservoir.

22.-24. (canceled)

25. A fluid delivery device, the fluid delivery device comprising:
a housing;
a reservoir disposed within the housing, the reservoir comprising an outlet port at a distal end, and plunger movable along a longitudinal axis of the reservoir, the plunger configured to provide a seal with respect to inner walls of the reservoir;
a plunger driver, the plunger driver comprising:
an inner screw being configured to advance the plunger along the longitudinal axis of the reservoir;
an outer screw concentrically surrounding the inner screw, wherein an inner surface of the outer screw engages with an outer surface of the inner screw such that a rotational movement of the outer screw advances the inner screw from a nested configuration to an extended configuration to move the plunger toward the distal end of the reservoir;
a screw support configured to be inserted into the reservoir at a proximal end of the reservoir and configured to engage with the outer screw; and
an anti-rotation sleeve (ARS) engaged with the screw support and the inner screw, such that, when the outer screw is rotated, the inner screw is advanced without rotating;
a drive nut concentrically surrounding and engaging the outer screw, wherein the drive nut is configured to be driven by an actuator to rotate the outer screw; and
a fluid path comprising a proximal end in fluid communication with the outlet port of the reservoir and a distal end configured to be inserted into a patient.

26. The fluid delivery device of claim 25, wherein the fluid delivery device comprises a patch pump.

27. The fluid delivery device of claim 25, wherein the fluid delivery device comprises a medication pen.

28. The fluid delivery device of claim 25, wherein the actuator comprises a motor.

29.-38. (canceled)
